(19) United States
(12) Patent Application Publication
(10) Pub. No.: US 2026/0273131 A1
(43) Pub. Date: Sep. 17, 2026

(54) Cell graft delivered in a delivery system and use thereof in regenerative medicine

(71) Applicant: China Medical University, Taichung City (TW)

(72) Inventors: Shih-Chieh HUNG, Taichung City (TW); Chun-Te HO, Taichung City (TW)

(21) Appl. No.: 19/116,844

(22) PCT Filed: Sep. 29, 2023
(86) PCT No.: PCT/US2023/075597

§ 371 Date: Mar. 28, 2025

Related U.S. Application Data
(60) Provisional application No. 63/412,055, filed on Sep. 30, 2022.

(51) Int. Cl. A61L 27/22; A61L 27/38; C07K 14/00

(52) U.S. Cl. CPC A61L 27/227; A61L 27/3834; C07K 14/001; A61L 2430/06; A61L 2430/16; A61L 2430/24; A61L 2430/38

(57) ABSTRACT

The present invention relates to a cell graft delivery system comprising a cell graft bound to a novel bifunctional peptide (BiFP), with tissue targeting function and cell differentiation instruction function, for delivering the cell graft to a target tissue. The bifunctional peptide (BiFP) of present invention is constructed with a peptide comprising collagen XII-targeting sequence mediating the tissue targeting function and an integrin/DDR binding motif sequence mediating the cell binding and cell differentiation instruction function.

### REFERENCE TO AN ELECTRONIC SEQUENCE LISTING

The content of the electronic sequence listing (5382-0363PWO2.xml; Size: 13 KB; and Date of Creation: 2024 Feb. 26) is herein incorporated by reference in its entirety.

### BACKGROUND OF THE INVENTION

### Technical Field of the Invention

The present invention relates to a cell graft and its delivery system for regenerative medicine. More particularly, it relates to a use of a stem cell graft delivered in a delivery system constructed with a bivalent peptide comprising collagen-like peptides (CLPs) with tissue-targeting and cell-fate-determining functions.

### Background

Collagen, the most abundant extracellular matrix (ECM) protein in the body, has been widely used in biomedical research and clinical practice. However, animal-derived collagen is limited due to its high immunogenicity and pathogenicity (Lynn, A. K., et al. J Biomed Mater Res B Appl Biomater 71, 343-354, 2004). Instead, the development of synthetic and performance- and structure-optimized collagen-like materials derived from supramolecular assembly of building blocks, such as collagen-like peptides (CLPs), has been applied in 3D cell culture and tissue engineering (Prince, E. & Kumacheva, E. Nature Reviews Materials 4, 99-115, 2019). The fibrous structure of these materials mimics the filamentous structure of the ECM, determining their biomechanical properties, signal transduction functions, and cellular instruction cues (Chau, M., et al. Advances in Polymer Science 268, 167-208, 2015). Besides fibrillar morphology and biophysical properties, the organization of adherent ligands also affects the way cells interact with these synthetic materials, and may accelerate regenerative medicine (Boekhoven, J. & Stupp, S. I. Adv Mater 26, 1642-1659, 2014). Another contribution that synthetic collagen-like materials should provide for successful cell therapy and regenerative medicine is the homing of transplanted cells into target tissues, such as those used for controlled drug delivery or genome editing technologies (Li, J. & Mooney, D. J. Nature Reviews Materials 1, 16071, 2016; Tong, S., et al. Nature Reviews Materials 4, 726-737, 2019). However, this is an unmet need in the field.

In the present invention, it is developed a bifunctional peptide (BiFP) for osteoarthritis (OA) and corneal epithelial defect regenerative medicine, which is a CLP with OA cartilage- and corneal stroma-targeting and cell fate determination functions. The OA cartilage- and corneal stroma-targeting function is mediated by a specific peptide sequence targeting collagen XII, an extracellular matrix that is uniquely expressed in OA cartilage and corneal stroma, while the cell differentiation instruction function is mediated by an integrin or DDR binding motif, which has previously been known to induce chondrogenic differentiation of human MSCs.

In our previous patent application TW202128730, it is showed that a collagen XII-targeting peptide (Col12-TP) can deliver MSCs to a damaged articular cartilage when conjugated with hyaluronic acid (HA). However, this approach requires HA being methacrylated with an incorporation rate of approximately 28%, and then conjugated with the Col12-TP by Michael addition chemistry. The MSC then binds to HA through the expression of CD44 on its surface. This strategy requires high chemical conjugation efficiency and depends on the expression of CD44.

Therefore, the present invention attempts to design a bifunctional peptide (BiFP) comprising an integrin or DDR binding motif flanked with CLP sequence triads and a Col12-TP sequence, to deliver a cell graft to a target tissue when binds to the cell graft.

### SUMMARY OF INVENTION

Based on the above object, the present invention demonstrates that the designed BiFP can bind to MSCs, and delivery MSCs to a target tissue, such as the surface of OA cartilage and corneal stroma for OA and corneal epithelial regeneration. Furthermore, the BiFP binding enhances viability, proliferation and chondrogenic differentiation of MSCs in a dose-dependent manner.

Accordingly, one aspect of the present invention relates to a cell graft delivered in a delivery system or a cell graft composition, comprising a cell graft binding to a bifunctional peptide (BiFP) for delivering the cell graft to a target tissue, wherein the BiFP is consisted of a tissue-targeting sequence and a cell-binding motif sequence flanked by GPO repeats. In some embodiments, the target tissue expresses collagen XII, including osteoarthritis (OA) or degenerated disc, corneal epithelium, infarcted cardiac tissue, skin dermis and tissue around hair follicle.

In some embodiments, the tissue-targeting sequence is an osteoarthritis (OA) or degenerated disc-targeting sequence. In some embodiments, the cell-binding sequence is a binding motif of an integrin or DDR.

In some embodiments of the present invention, the bifunctional peptide comprises a collagen XII-targeting sequence, an integrin α2β1- or DDR-binding motif sequence and at least three copies of GPO flanking the integrin- or DDR-binding motif.

In some embodiments of the present invention, the integrin-motif is an α2β1 integrin binding motif. In one embodiment, the α2β1 integrin binding motif has a sequence of GFOGER.

In other embodiments of the present invention, the cell-binding motif is a DDR binding motif. In one embodiment, the DDR binding motif has a sequence of GVMGFO.

In one embodiment, the bifunctional peptide has a sequence of GPOGPOGPOGPOGFOGERGPOGPOGPOGPODLQYWYPIWDTH. In another embodiment, the bifunctional peptide has a sequence of GPOGPOGPOGPOGVMGFOGPOGPOGPOGPODLQYWYPIWDTH.

In some embodiments of the present invention, the cell graft includes, but not limited to, MSC, progenitor or differentiated cell of musculoskeletal system, and corneal cell or its progenitor cell. In one embodiment, the cell graft is an autologous MSC graft. In other embodiment, the cell graft is an allogenous MSC graft.

In another aspect of the present invention, it is related to a method for treating a disease involving collagen XII expression, comprising administering a subject in need thereof with a therapeutically effective amount of a cell graft delivered with a bifunctional peptide (BiFP), wherein the BiFP is consisted of a tissue-targeting sequence and a cell-binding motif sequence flanked by GPO repeats. In some embodiments of the present invention, the disease involving collagen XII expression includes, but not limited to, a musculoskeletal disorder, a septic or aseptic keratitis, dry eye syndrome, a cardiac disease, a skin defect and a disease involving hair follicle.

In some embodiments of the present invention, the musculoskeletal disorder includes sprains, strains and tears of ligaments, tendons, muscles and cartilage, tendonitis, tenosynovitis, fibromyalgia, osteoarthritis, rheumatoid arthritis, intervertebral disc disease, polymyalgia rheumatica, bursitis, acute and chronic back pain, osteoporosis, carpal tunnel syndrome, De Quervains's disease, trigger finger, tennis elbow, rotator cuff, ganglion cysts, osteogenesis imperfecta, Duschennes, Hurler's and Hunter's syndromes and combination thereof. In one embodiment, the musculoskeletal disorder is osteoarthritis (OA).

In other embodiments of the present invention, the cardiac disease is a myocardial infarction. In other embodiments of the present invention, the skin defect is a burn injury.

A further aspect of the present invention relates to a cell graft composition, comprising a cell graft delivered with a bifunctional peptide (BiFP), wherein the BiFP is consisted of a tissue-targeting sequence and a cell-binding motif sequence flanked by GPO repeats.

In some embodiments of the present invention, the cell graft composition induces regeneration of neocartilage in an OA knee joint. In some embodiments of the present invention, the cell graft composition induces corneal epithelial regeneration in a corneal epithelium defect area.

### BRIEF DESCRIPTION OF THE DRAWINGS

FIG. 1a shows the circular dichroism (CD) spectra of peptides with representative secondary structures. The triple helix structure of BiFP, collagen, or denatured collagen peptides was measured at 0.2 mg/ml. FIG. 1b shows the particle size distribution of 0.2 mg/ml BiFP in PBS at 25° C. which is examined by dynamic light scattering (DLS). Data revealed uniform size distribution (~1.914 nm).

FIG. 2a and FIG. 2b show the results of the analysis of hMSCs binding to BiFP. The hMSCs were labeled with Hochest 33258, and incubated with FITC-labeled BiFP for 1 h, followed by imaging flow cytometry on an Amnis Imaging Cytometer (left). Bar=10 μm. Quantification of the percentage of BiFP-bound hMSCs (right).

FIG. 3a shows the measurement of cell viability of human mesenchymal stem cells (hMSCs) treated with collagen XII-targeting peptide (Col12-TP) and BiFP at indicated concentrations. The hMSCs are incubated with Col12-TP or BiFP peptide for 1 h, and stained with Calcein-AM (green) and ethidium homodimer (magenta), followed by confocal microscopic observation (left). Bar=50 μm. The quantification of viable cell percentages (right). FIG. 3b shows the growth curves of Col12-TP- or BiFP-bound hMSCs are analyzed by WST-1 proliferation assay. FIG. 3c shows the Col12-TP- or BiFP-bound hMSCs are analyzed for indicated mRNA levels by real-time RT-PCR. FIG. 3d shows the Measurement of sulfated glycosaminoglycan (sGAG) production in Col12-TP- or BiFP-bound hMSCs at 14 days. The hMSCs are stained with 1,9 dimethylmethlyene blue (DMB) dye and analyzed at OD 525 nm. Results are normalized by PicoGreen dsDNA quantitation. The data are shown as mean±SD. * p<0.05, ** p<0.01, *** p<0.001 and **** p<0.0001 as determined by Student's t test (2 groups) or the ANOVA (≥3 groups).

FIG. 4a and FIG. 4b show the FITC-fluorescence-labeled BiFP without or with blocking peptide (FIG. 4a) and the DilC18-fluorescence-labeled rat MSCs (rMSCs) without or with delivery in BiFP (BiFP+rMSCs) (FIG. 4b) are intra-articularly injected into rat OA knee joints pre-established with papain/cysteine, respectively. The whole joint capsules are removed at 24 h post-injection, and articular surfaces of the distal femur are observed under a two-photon microscope. Representative images of second harmonic generation (SHG) signals in collagen and fluorescent signals of BiFP (FIG. 4a) and rMSCs (FIG. 4b) are shown (left). Quantification of overall fluorescence areas (right). FIG. 4c and FIG. 4d show the representative images of Hematoxylin and eosin (H&E) staining (FIG. 4c), and safranin-O/fast green staining (FIG. 4d) of distal femurs of sham-control or OA knee joints collected 8 weeks post-transplantation without or with BiFP, rMSCs, and rMSCs delivered in BiFP (BiFP+rMSCs). FIG. 4e shows the result of analysis of OA degrees with modified Mankin's scores according to slides from H&E and safranin-O/fast green staining. Bar=50 μm. The data are shown as mean±SD. * p<0.05, ** p<0.01 and **** p<0.0001 as determined by Student's t test (2 groups) or one-way ANOVA (≥3 groups).

FIG. 5a is the schematic flowchart of the experimental design for regenerating multilayered articular cartilage by EGFP-expressing human MSCs without or with delivery in BiFP (BiFP+hMSCs) in the rat osteoarthritis (OA) model induced by papain/cysteine. The rats were sacrificed at indicated time point. The distal femurs of knee joints were subjected to immunohistochemistry or immunofluorescence analysis. FIG. 5b shows the immunohistochemistry for EGFP. Positive signal is visualized as brown color (DAB) and NC is a negative control without first antibody. Bar=50 μm. FIG. 5c shows the immunofluorescence of EGFP, aggrecan, collagen 2 and collagen 12 performed on rat OA cartilage treated with EGFP-hMSCs or BiFP-delivered EGFP-hMSCs to assess expression of chondrogenic markers. Bar=50 μm.

FIG. 6a is the schematic flowchart of the experimental design for regenerating multilayered articular cartilage by EGFP-expressing rMSCs without or with delivery in BiFP (BiFP+rMSCs) in the rat osteoarthritis (OA) model induced by papain/cysteine. The rats were sacrificed at indicated time point. The distal femurs of knee joints were subjected to immunohistochemistry or immunofluorescence analysis. FIG. 6b shows the immunohistochemistry for EGFP. Positive signal is visualized as brown color (DAB). FIG. 6c shows the double immunofluorescence of EGFP and the expression of chondrogenic markers, such as aggrecan, collagen 2 or collagen 12. Cell nuclei were counterstained with 4′, 6-diamidino-2-phenylindole (DAPI). Bar=50 μm.

FIG. 7a is the schematic flowchart of the experimental design for intervertebral disc (IVD) regeneration by PBS, BiFP, EGFP-expressing hMSCs without or with delivery in BiFP (BiFP+hMSCs) in the rat intervertebral disc damage model induced by 20 G needle puncture. The rats were sacrificed at the indicated time point. The rat tail tissues were subjected to X-Ray, T2-weighted MRI, Hematoxylin and eosin (H&E), Safranin O/fast green stain, immunohistochemistry and immunofluorescence analysis. FIG. 7b shows the radiographic images of bony structure, while FIG. 7c shows the T2-weighted MRI images of nucleus pulposus (NP) in the coccygeal joints that were subjected to intervertebral injury (Sham group was not subjected to intervertebral injury), followed by different treatments two weeks later, and the specimens were harvested for radiographical analysis. FIG. 7d and FIG. 7e show the representative images of H&E staining and safranin-O/fast green staining of NP tissues, respectively. FIG. 7f shows the immunohistochemistry for EGFP. Positive signal is visualized as brown color (DAB). FIG. 7g shows the double immunofluorescence of EGFP and the expression of chondrogenic markers, such as aggrecan or collagen 2. Cell nuclei were counterstained with 4′, 6-diamidino-2-phenylindole (DAPI). Bar=50 μm.

FIG. 8a is the schematic flowchart of the experimental design for corneal epithelium regeneration by EGFP-expressing hMSCs without or with delivery in BiFP (BiFP+hMSCs) in the rat corneal epithelium damage model. FIG. 8b shows corneal epithelial defects (CED) induced by n-heptanol injury were stained with fluorescein at the indicated times after the onset of treatment with one eye drop (20 μl) containing PBS, BiFP (40 M), EGFP-hMSCs (105), or BiFP-delivered EGFP-hMSCs. Representative fluorescein-stained cornea images are shown at indicated time points (left). Quantification of the percentage of healed area was calculated using ImageJ Fiji software (right). * p<0.05, ** p<0.01 and *** p<0.001 as determined by One Way ANOVA. FIG. 8c shows the histomorphology of cornea in each group at 14 days was detected by H&E stain. Bar=50 μm. FIG. 8d shows the immunohistochemistry for EGFP. Positive signal is visualized as brown color (DAB) and NC is negative control. Bar=50 μm.

### DETAILED DESCRIPTION OF THE INVENTION

Other features and advantages of the present invention will be further exemplified and described in the following examples, which are intended to be illustrative only and not to limit the scope of the invention.

### Example 1: Preparation of a Cell Graft Delivery System Comprising a Bifunctional Peptide (BiFP)

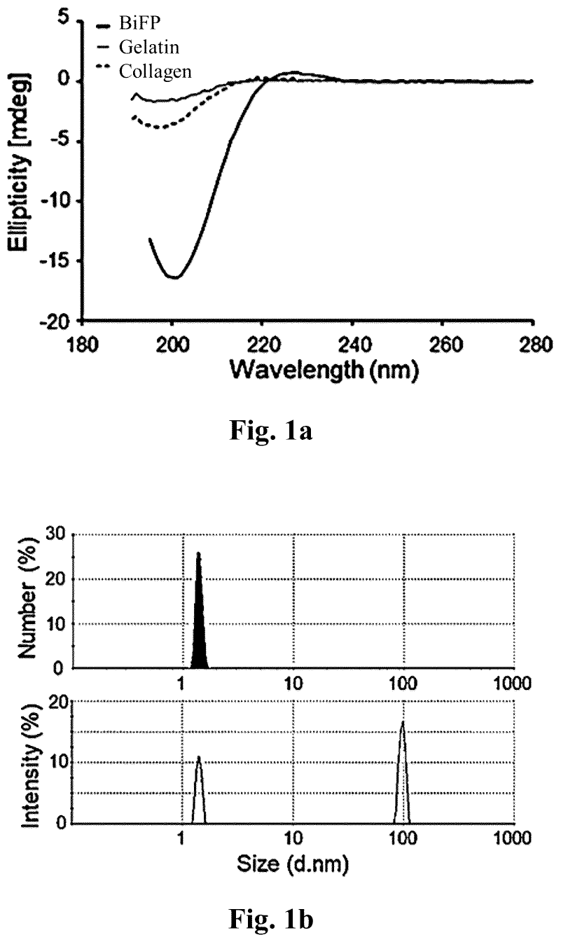

In the present invention, a bifunctional peptide (BiFP) is synthesized to prevent complicated chemical steps and improve efficiency. A 42-residue peptide (BiFP) with a sequence where GPOGPOGPOGPOGFOGERGPOGPOGPOGPODLQYWYPIWDTH, O is hydroxyproline, was prepared in PBS, pH 7.4 at 25° C. by Biomedical Translation Research Center (Taiwan). The triple helix structure of BiFP, collagen, or denatured collagen peptides was measured at 0.2 mg/ml, respectively. The particle size distribution of 0.2 mg/ml BiFP in PBS at 25° C. was examined by dynamic light scattering (DLS). Circular dichroism (CD) spectroscopy verified that this peptide adopts a stable triple-helical conformation in solution (FIG. 1a). Size was evaluated using dynamic light scattering: Malvern ZS90 zetasizer (Malvern Instruments Corp, Malvern, UK). The dynamic light scattering data revealed uniform size distribution and the main size is 1.914 nm in PBS (FIG. 1b).

The FITC-labeled BiFP or rhodamine-labeled collagen XII-targeting peptide (Col12-TP) are chemically synthesized by Biomertech (USA) or ABI (USA) to identify peptide binding by confocal microscopic observation. Briefly, for FITC-BiFP or rhodamine-Col12-TP treatment, 1×10\hat{ }6 hMSCs incubated with 1 μM DilC18 in 37° C. for 10 min and then incubated in final 200 μl PBS with fluorescent peptide at 0, 2.5, 10, 40 μM in 37° C. for 30 min with gently mixing every 10 min. Cells are counterstained with mounting solution containing DAPI. Peptide fluorescence was taken using an ImageXpress Micro Confocal High Content Imaging system (Molecular Devices, Sunnyvale, CA, USA).

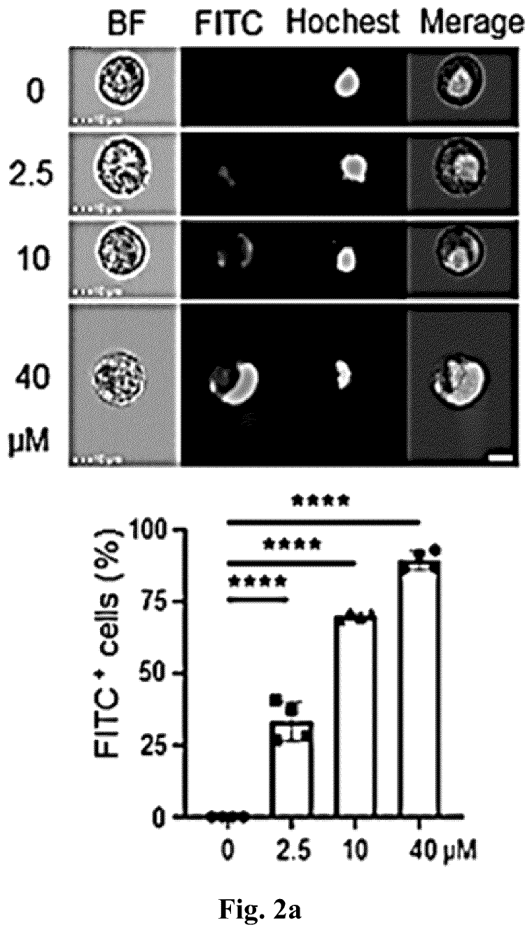

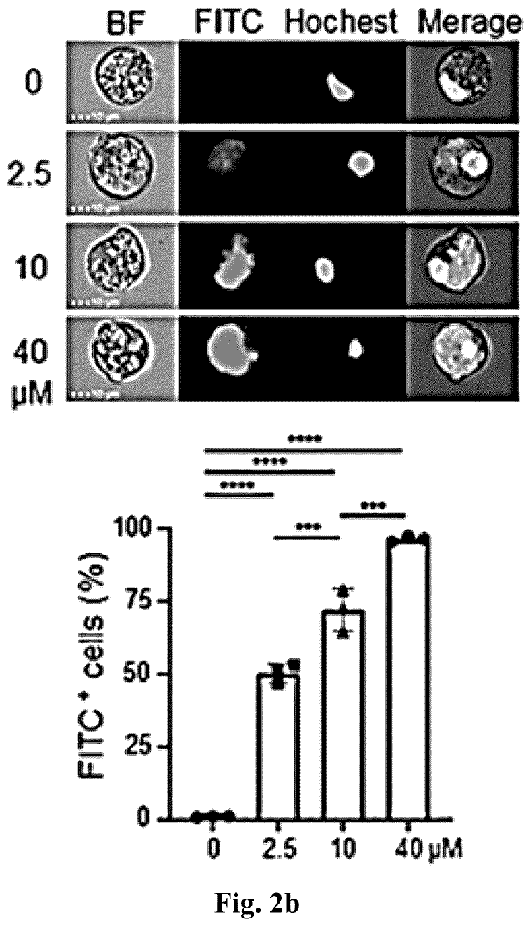

We first show that Col12-TP cannot bind to hMSCs, while BiFP can bind to hMSCs in a dose-dependent manner. Imaging flow cytometry shows data similar to microscopic observation and further reveals that increasing the concentration of BiFP from 2.5 to 40 μM results in an increase in the percentage of cells surrounded by FITC-conjugated BiFP (FIG. 2a). Consistently, hMSCs do not express collagen XII, while the chondrocyte line hiP cells express collagen XII and can bind to Col12-TP.

Besides, another 42-residue peptide (BiFP) with a sequence GPOGPOGPOGPOGVMGFOGPOGPOGPOGPODLQYWYPIWDTH, where O is hydroxyproline, is also prepared, in which a CLP containing a GVMGFO sequence, the DDR binding motif, flanked by GPO repeats i

s incorporated into a collagen XII-targeting peptide. As showed in FIG. 2b, the BiFP can also bind to hMSCs. Similarly, imaging flow cytometry shows that increasing the concentration of BiFP from 2.5 to 40 μM results in an increase in the percentage of cells surrounded by FITC-conjugated BiFP (FIG. 2b). These data suggest that a bifunctional peptide (BiFP) containing a GVMGFO sequence, the DDR binding motif can also be designed to develop a cell graft delivery system for regeneration.

### Example 2: BiFP Binding Enhances MSC Viability, Proliferation and Chondrogenic Differentiation

For BiFP or Col12-TP treatment, 1×10\hat{ }6 hMSCs incubated in final 200 μl PBS with peptide at 0, 2.5, 10, 40 μM in 37° C. for 30 mins with gently mixing every 10 min. Cells are stained with Calcein-AM (ThermoFisher) for live hMSCs and ethidium homodimer (Life Technologies) for dead hMSCs, respectively. Labeled hMSCs are visualized with ImageXpress Micro Confocal High Content Imaging system (Molecular Devices, Sunnyvale, CA, USA). Three independent images each group are counted live hMSCs and quantified.

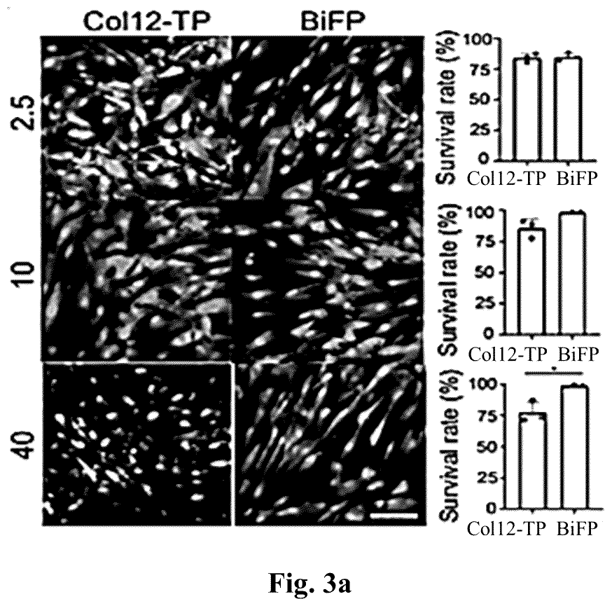

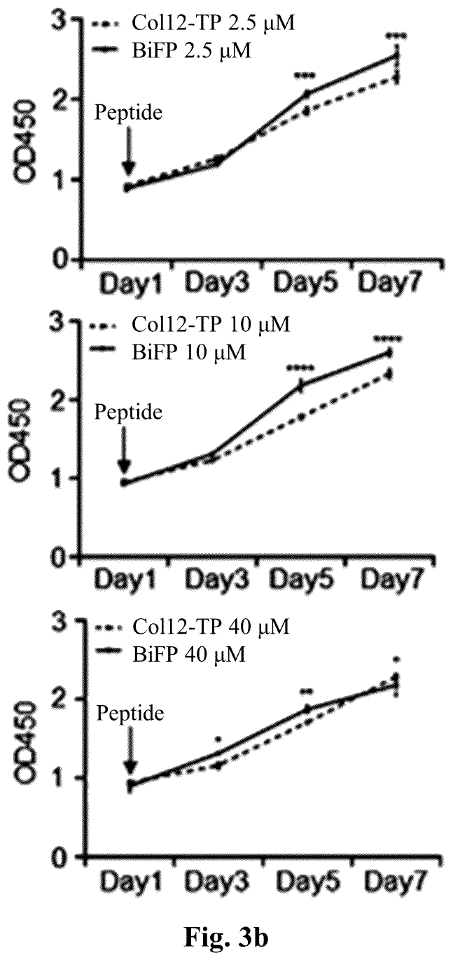

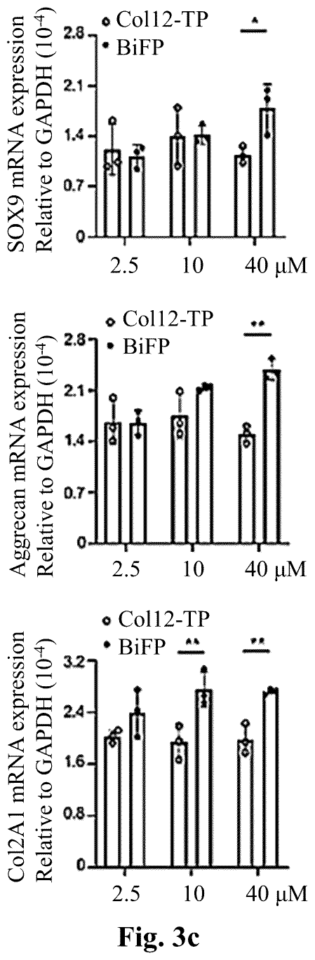

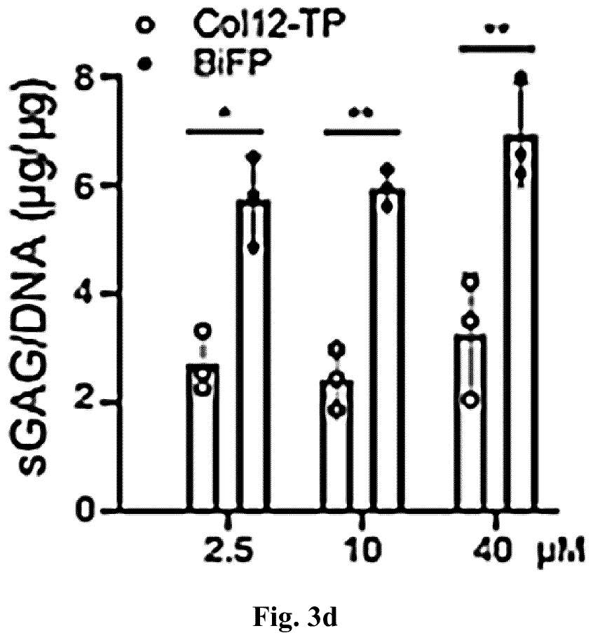

Compared to Col12-TP, human MSCs incubated with BiFP increase cell viability (FIG. 3a), cell proliferation (FIG. 3b) and differentiation into chondrogenic cells as shown by the increased expression of chondrogenic genes, such as sox9, col2a1 and aggrecan at 1 week (FIG. 3c), and enhance synthesis of glycosaminoglycan at 2 weeks (FIG. 3d). To be noted, the effects of BiFP on viability, proliferation are more obvious in higher concentration than lower concentration (FIG. 3). Together, these data suggest BiFP binding enhances MSC viability, proliferation, and chondrogenic differentiation in a dose-dependent manner.

### Example 3: Application of the BiFP Comprising Cell Graft Delivery System in OA Regenerative Medicine

Since BiFP can home to OA cartilage and the chondrogenesis-inducing ability of integrin α2β1 is likely to induce MSCs to undergo chondrogenesis, the BiFP is applied in the delivery of human MSCs to the OA cartilage surface for OA regenerative medicine. To demonstrate the OA-specific targeting activity of BiFP, rhodamine-labeled BiFP without or with preincubation with collagen XII blocking peptide are separately injected into OA joints in a rat model for two-photon microscopy observation of fluorescence and second harmonic generation (SHG) signals.

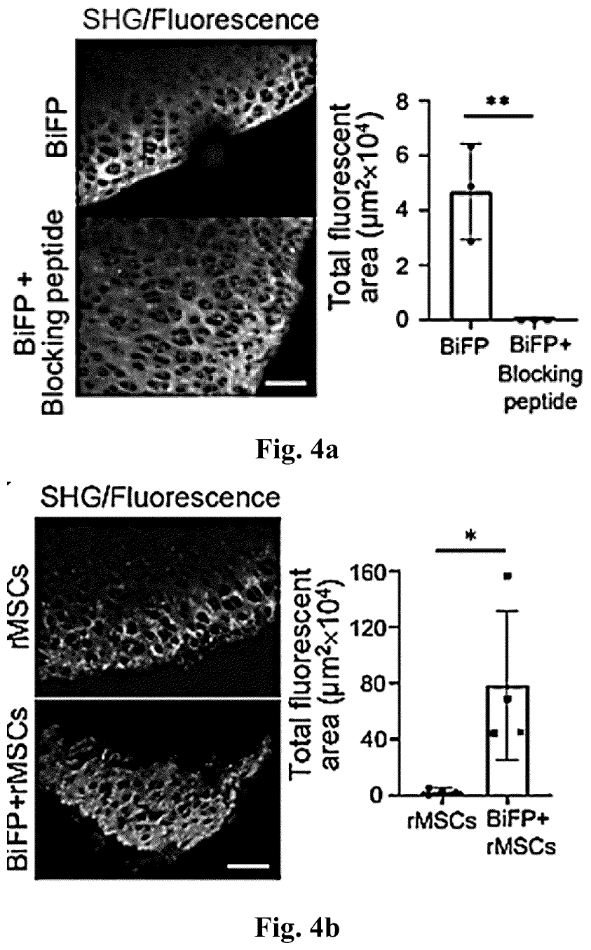

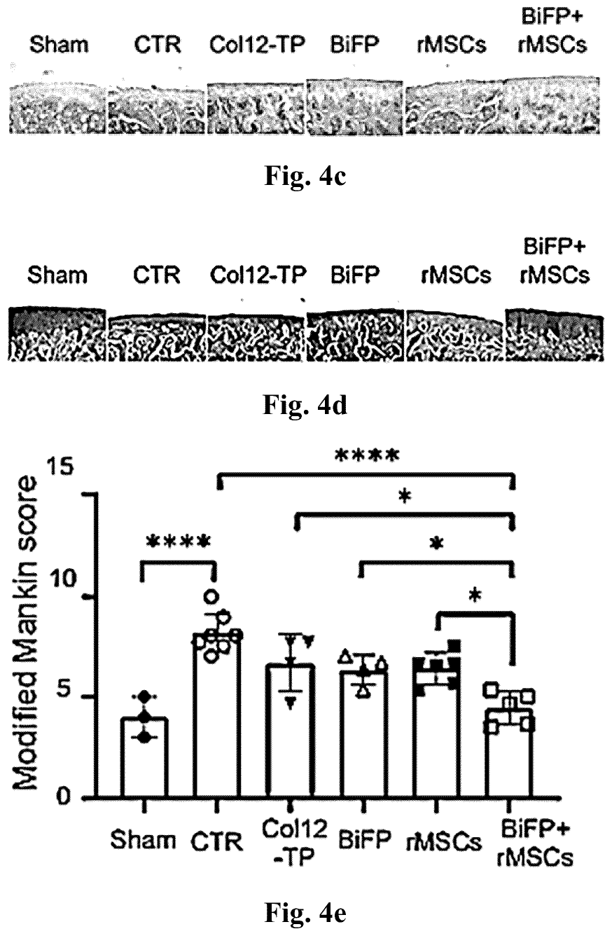

Surface-rendered 3D reconstructed images and transversal composite images of cartilage showed apparent red dots are observed in the BiFP-injected OA cartilage (FIG. 4a). When probing type II collagen with SHG, red dots are localized in the SHG signal-free area, corresponding to the territorial region (pericellular region) of OA cartilage. Conversely, red dots are not observed in the collagen XII blocking peptide-preincubated BiFP-injected OA cartilage (FIG. 4a).

MSCs are then labeled with Dil viable dye before incubated without or with BiFP, followed by intraarticular injection into the rat OA knee joints. When probing type II collagen with SHG, red dots are observed on the articular cartilage surface in rat OA knees that received MSCs incubated with BiFP, but not in that received MSCs without incubation with BiFP (FIG. 4b).

Furthermore, after incubation with BiFP, MSCs are immediately injected into OA joints in a rat model, and the joints are subjected to histological examination 8 weeks post-transplantation. Histomorphometric analysis reveals the successful induction of OA when compared OA group with sham control group (FIG. 4c, 4d). Moreover, knee joints receiving MSCs delivered by BiFP had apparent cartilage regeneration and safranin-O staining (FIG. 4c, FIG. 4d), while those receiving MSCs without delivery in BiFP, Col12-TP alone, and BiFP alone still exhibited severe OA, showing multiple cracks on the surface of the cartilage with the loss of safranin-O staining.

Quantification of OA degree by modified Mankin score also revealed that sham control group had reduced OA score than OA group (FIG. 4e). Similarly, OA treated with MSCs delivered in BiFP significantly improved in modified Mankin score than OA treated with MSCs without delivery in BiFP, Col12-TP alone, or BiFP alone (FIG. 4e).

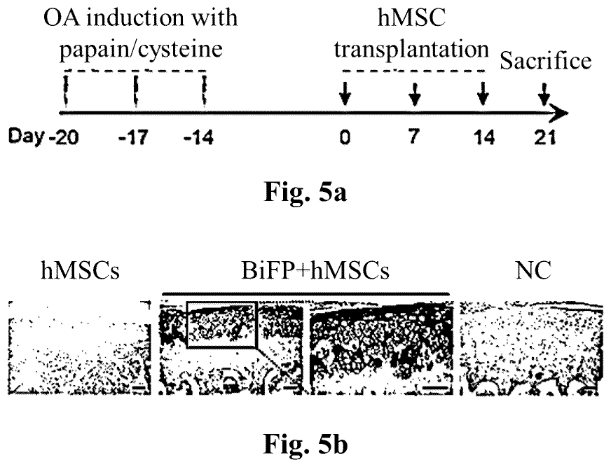

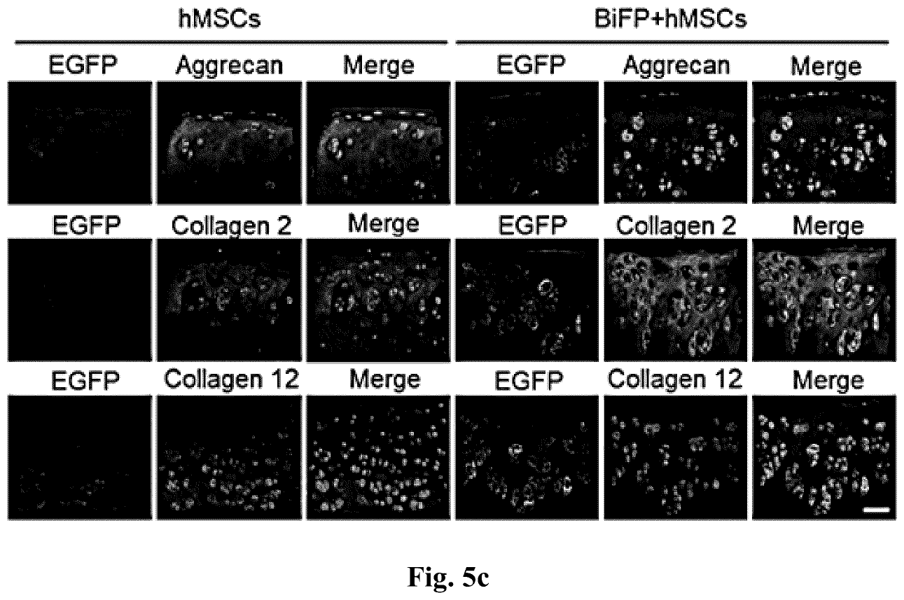

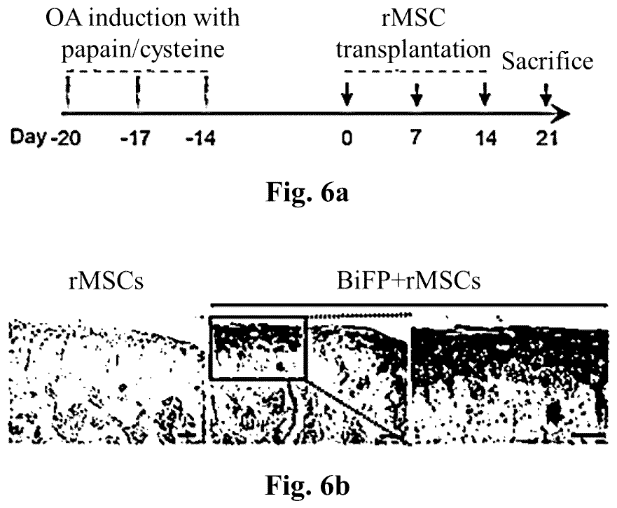

Human and rat MSCs are then lentivirally transduced with EGFP for long-term tracking, and then delivered in BiFP and intra-articularly injected into the OA knee joints of the rat osteoarthritis (OA) model once a week once a week for 3 weeks (FIG. 5a, FIG. 6a). Seven days after the third transplantation, the knee joints are harvested for cell fate determination by histological evaluation of chondrogenic protein expression of the transplanted MSCs.

Since MSCs have the immune privileged nature, EGFP-human MSCs (EGFP-hMSCs) delivered in BiFP are intra-articularly injected into the rat OA knee joints once a week for 3 weeks (FIG. 5a), followed by histological assessment of cartilage regeneration one week later. Remarkably, it is observed that multiple layers of GFP+ neocartilage are arranged in three vertical blocks, with cells in the lower and middle blocks having mature chondrocyte-like spherical shape, and cells in the upper block having MSC-like fibroblast shape (FIG. 5b). Furthermore, these cells are positive for aggrecan and collagen II (FIG. 5c). Together, these data suggest multiple injection of rat and human MSCs delivered in BiFP regenerate multi-layers of neocartilage.

FIG. 6 shows the application of multiple intra-articular injections of bi-functional peptide (BiFP)-delivered rat MSCs (rMSCs) in osteoarthritis regenerative medicine. Immunostaining reveals multi-layers of EGPF+, aggrecan+ and collagen II+ cells in knee joints receiving EGFP-rat MSCs (rMSCs) delivered in BiFP, but not in that receiving EGFP-rMSCs without delivery in BiFP (FIG. 6b). Furthermore, double immunofluorescence reveals colocalization of EGFP with aggrecan and collagen II (FIG. 6c). Notably, multi-layers of collagen XII+ cells are observed in knee joints that receiving EGFP-rMSCs without delivery in BiFP, while only single layer or sparse collagen XII+ cells are observed in knee joints receiving EGFP-rMSCs delivered in BiFP.

### Example 4 Application of the BiFP Comprising Cell Graft Delivery System in Intervertebral Disc Regeneration

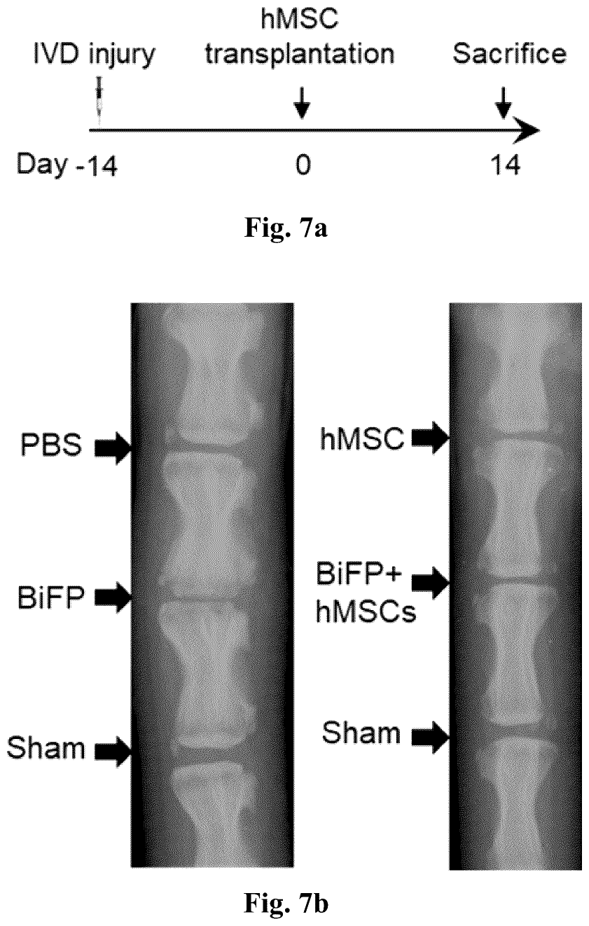

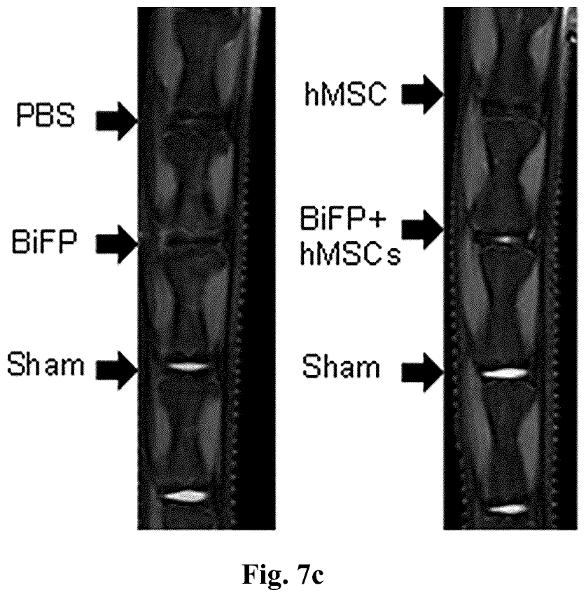

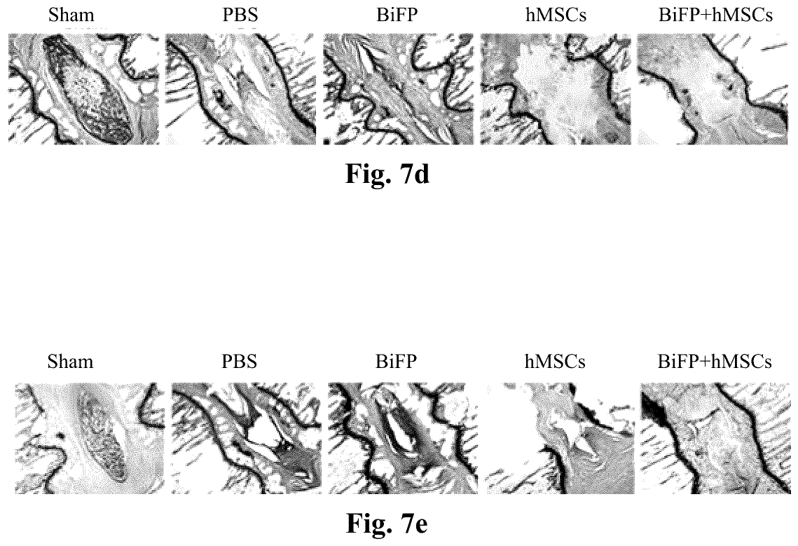

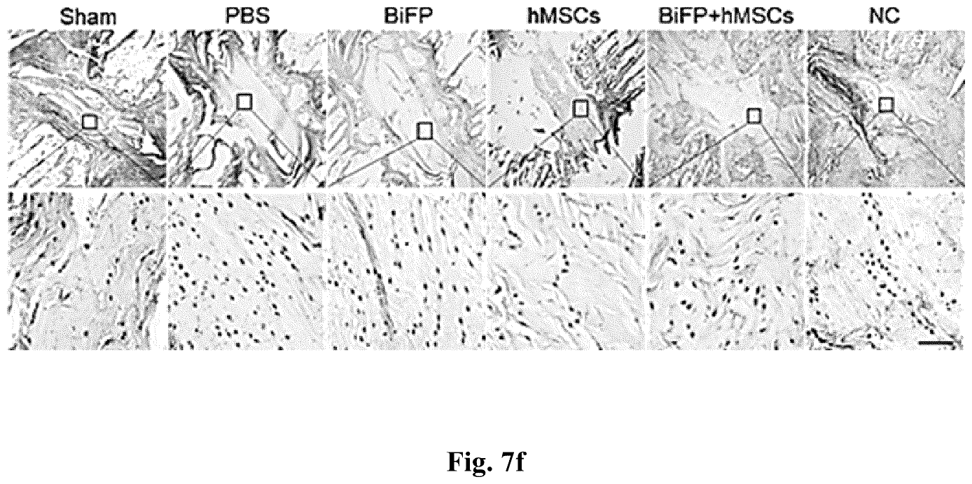

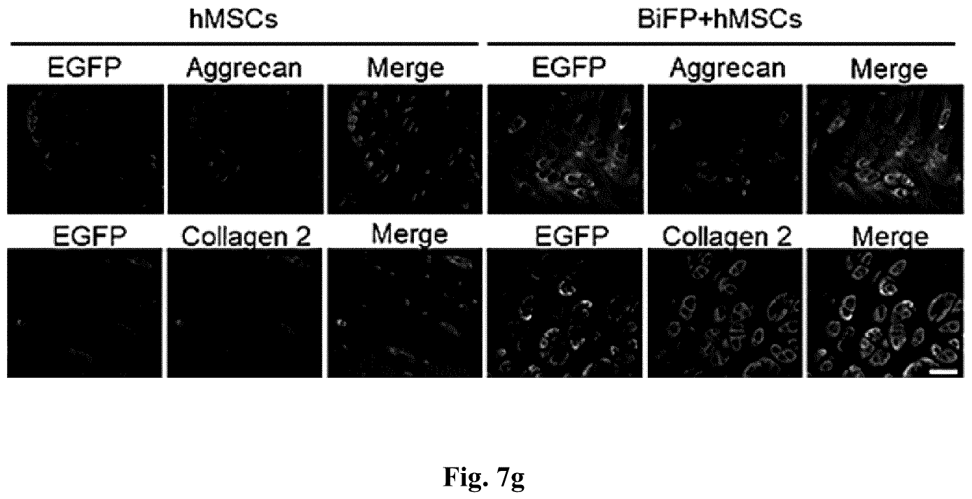

FIG. 7 shows the application of bi-functional peptide (BiFP)-delivered human MSCs (hMSCs) in intervertebral disc (IVD) regeneration. X-ray, T2-weighted MRI and histological, histochemical and immunofluorescence analyses reveal the successful induction of IVD damage when compared PBS group with sham control group (FIG. 7b-7e). Moreover, nucleus pulposus receiving hMSCs delivered by BiFP preserved the IVD height (FIG. 7b) had apparent intervertebral disc regeneration as evident by T2 high water-containing disc (FIG. 7c), safranin-O positive nucleus pulposus (FIG. 7d), while those receiving MSCs without delivery in BiFP, BiFP alone, and PBS still exhibited severe IVD damage with consequent loss of disc height and T2 high water-containing disc, and exhibit the loss of safranin-O staining. Immunostaining reveals multi-layers of EGPF+, aggrecan+ and collagen II+ cells in nucleus pulposus receiving EGFP-hMSCs delivered in BiFP, but not in that receiving EGFP-hMSCs without delivery in BiFP (FIG. 7f, 7g).

### Example 5: Application of MSCs Delivered in the BiFP Comprising Cell Graft Delivery System for Corneal Epithelial Regeneration

Collagen XII is expressed in a variety of tissues including the cornea, which is abundant in the stroma and the anteriorly located Bowman's layer, and is exposed when the eye has corneal epithelial defect. When disease has affected corneal clarity, corneal transplantation remains the main method for visual rehabilitation. In addition, cadaveric corneal epithelial stem cells are transplanted with amniotic membrane carriers for severe ocular surface disease and limbal dysfunction. However, all these techniques depend on the availability of corneal donor tissue, which is the major limiting factor in developing countries.

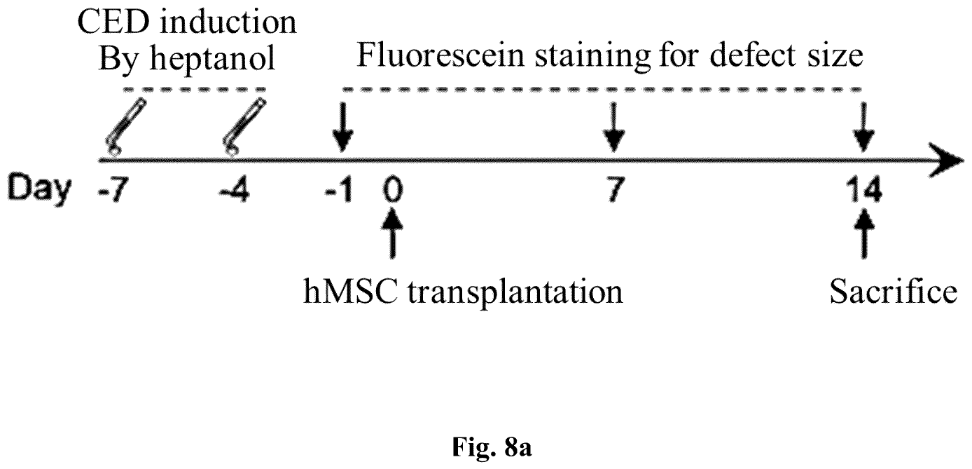

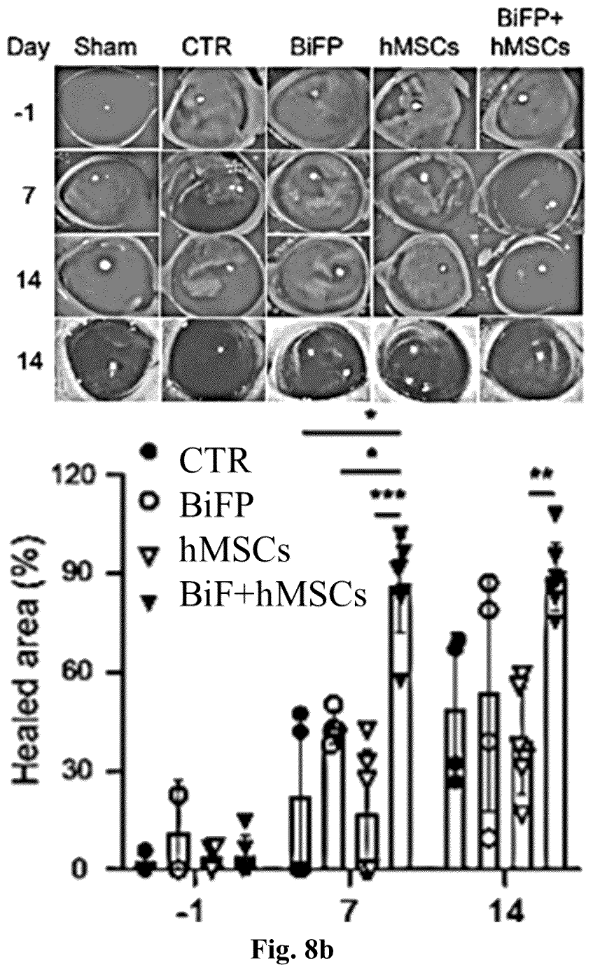

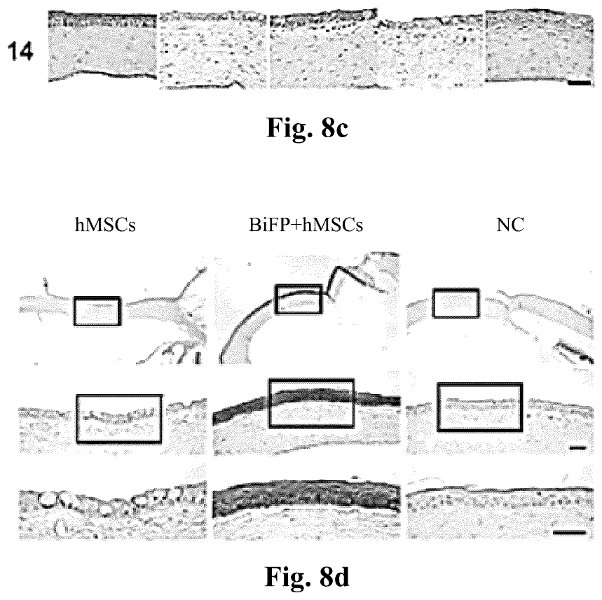

To further confirm the utility of BiFP as a cell carrier and expand the application of BiFP-delivered MSCs in corneal epithelial defect regeneration, a rat model of severe and corneal epithelial injury induced by repeated n-heptanol administration was utilized. Measurement of corneal defect area by sodium fluorescein staining revealed that administration of n-heptanol twice within four days resulted in non-healing of the corneal epithelium after two weeks, suggesting that this is a critical model of corneal epithelial injury (FIG. 8a, flow chart).

Interestingly, corneas that received hMSCs delivered in the BiFP comprising cell graft delivery system are free of defects one week after corneal injury, whereas corneas that received hMSCs or BiFP alone remained markedly defective even 2 weeks after injury (FIG. 8b). Histomorphological analysis showed the same results that hMSCs delivered by BiFP significantly improved corneal injury healing compared to hMSCs or BiFP alone (FIG. 8c).

Immunohistochemistry showed that all neo-epithelial cells are GFP+, indicating that hMSCs engrafted and became neo-epithelial layers in the damaged cornea (FIG. 8d). Notably, the neoepithelial morphology formed by hMSCs had the morphology of normal corneal epithelium, with flat cells in the upper layer and round cells in the lower layer (FIG. 8d). These data suggest that BiFP acts as a cellular carrier for the delivery of hMSCs in corneal epithelial regeneration.

In summary, the present invention first synthesizes a CLP containing a GFOGER sequence or a GVMGFO sequence flanked by GPO repeats and incorporated it into a collagen XII-targeting peptide, and thereby a bifunctional peptide (BiFP). The BiFP is then designed to develop a cell graft delivery system for regeneration. For instance, in cartilage regenerative medicine, the cell graft delivery system may solve the unmet need of injured or degenerative articular cartilage, and help repair or regenerate damaged or degenerated joints with layers of newly grown chondrocytes. Also in corneal regenerative medicine, hMSCs delivered by the cell graft delivery system significantly improve corneal injury healing in a corneal epithelial injury rat model. Therefore, the cell graft delivery system can also be applied to regeneration medicine for degenerative discs, corneal damage in keratitis or dry eye syndrome, and other diseases involving collagen XII expression, such as myocardial infarction, burn injury, and alopecia.

1. A cell graft delivery system, comprising a cell graft and a bifunctional peptide (BiFP) delivering the cell graft to a target tissue, wherein the cell graft is bound to the BiFP which is consisted of a tissue-targeting sequence and a cell-binding motif sequence flanked by GPO repeats.

2. The cell graft delivery system of claim 1, wherein the tissue-targeting sequence is an osteoarthritis (OA) or degenerated disc-targeting sequence.

3. The cell graft delivery system of claim 1, wherein the tissue-targeting sequence is a collagen XII-targeting sequence.

4. The cell graft delivery system of claim 1, wherein cell-binding sequence is a binding motif of an integrin or DDR.

5. The cell graft delivery system of claim 1, wherein the bifunctional peptide comprises a collagen XII-targeting sequence, an integrin α2β1- or DDR-binding motif sequence and at least three copies of GPO flanking the integrin- or DDR-binding motif.

6. The cell graft delivery system of claim 4, wherein the integrin-motif is an integrin α2β1 binding motif.

7. The cell graft delivery system of claim 6, wherein the α2β1 binding motif has a sequence of GFOGER.

8. The cell graft delivery system of claim 4, wherein the cell-binding motif is a DDR binding motif.

9. The cell graft delivery system of claim 8, wherein the DDR binding motif has a sequence of GVMGFO.

10. The cell graft delivery system of claim 1, wherein the bifunctional peptide has a sequence of GPOGPOGPOGPOGFOGERGPOGPOGPOGPODLQYWYPIWDTH.

11. The cell graft delivery system of claim 1, wherein the bifunctional peptide has a sequence of GPOGPOGPOGPOGVMGFOGPOGPOGPOGPODLQYWYPIWDTH.

12. The cell graft delivery system of claim 1, wherein the cell graft is selected from MSC, progenitor or differentiated cell of musculoskeletal system, and corneal cell or its progenitor cell.

13. The cell graft delivery system of claim 1, wherein the cell graft is an autologous MSC graft.

14. The cell graft delivery system of claim 1, wherein the cell graft is an allogenous MSC graft.

15. A method for treating a disease involving collagen XII expression, comprising administering a subject in need thereof with a therapeutically effective amount of a cell graft delivered with a bifunctional peptide (BiFP), wherein the bifunctional peptide is consisted of a tissue-targeting sequence and a cell-binding motif sequence flanked by GPO repeats.

16. The method of claim 15, wherein the disease involving collagen XII expression is a musculoskeletal disorder, a septic or aseptic keratitis, dry eye syndrome, a cardiac disease, a skin defect or a disease involving hair follicle.

17. The method of claim 16, wherein the musculoskeletal disorder includes sprains, strains and tears of ligaments, tendons, muscles and cartilage, tendonitis, tenosynovitis, fibromyalgia, osteoarthritis, rheumatoid arthritis, intervertebral disc disease, polymyalgia rheumatica, bursitis, acute and chronic back pain, osteoporosis, carpal tunnel syndrome, De Quervains's disease, trigger finger, tennis elbow, rotator cuff, ganglion cysts, osteogenesis imperfecta, Duschennes, Hurler's and Hunter's syndromes and combination thereof.

18. The method of claim 16, wherein the musculoskeletal disorder includes osteoarthritis (OA) and intervertebral disc disease (IVD).

19. The method of claim 16, wherein the cardiac disease is myocardial infarction.

20. The method of claim 16, wherein the skin defect is burn injury.

21. A cell graft composition for regenerative medicine, comprising a cell graft delivered with a bifunctional peptide (BiFP), wherein the bifunctional peptide is consisted of a tissue-targeting sequence and a cell-binding motif sequence flanked by GPO repeats.

22. The cell graft composition of claim 21, wherein the cell graft composition is used to induce neocartilage regeneration in an OA knee joint.

23. The cell graft composition of claim 21, wherein the cell graft composition is used to induce disc regeneration in a disc with IVD.

24. The cell graft composition of claim 21, wherein the cell graft composition is used to induce corneal epithelial regeneration in a corneal defect area.


SEQUENCE LISTING (4 sequences)

SEQ ID NO: 1 - protein, 6 aa, synthetic construct
  REGION 1..6: note = Synthetic: integrin binding motif
  MOD_RES 3: note = Hydroxyproline
GFPGER

SEQ ID NO: 2 - protein, 6 aa, synthetic construct
  REGION 1..6: note = Synthetic: DDR binding motif
  MOD_RES 6: note = Hydroxyproline
GVMGFP

SEQ ID NO: 3 - protein, 42 aa, synthetic construct
  REGION 1..42: note = Synthetic: bifunctional peptide
  MOD_RES 3: note = Hydroxyproline
  MOD_RES 6: note = Hydroxyproline
  MOD_RES 9: note = Hydroxyproline
  MOD_RES 12: note = Hydroxyproline
  MOD_RES 15: note = Hydroxyproline
  MOD_RES 21: note = Hydroxyproline
  MOD_RES 24: note = Hydroxyproline
  MOD_RES 27: note = Hydroxyproline
  MOD_RES 30: note = Hydroxyproline
GPPGPPGPPGPPGFPGERGPPGPPGPPGPPDLQYWYPIWDTH

SEQ ID NO: 4 - protein, 42 aa, synthetic construct
  REGION 1..42: note = Synthetic: bifunctional peptide
  MOD_RES 3: note = Hydroxyproline
  MOD_RES 6: note = Hydroxyproline
  MOD_RES 9: note = Hydroxyproline
  MOD_RES 12: note = Hydroxyproline
  MOD_RES 18: note = Hydroxyproline
  MOD_RES 21: note = Hydroxyproline
  MOD_RES 24: note = Hydroxyproline
  MOD_RES 27: note = Hydroxyproline
  MOD_RES 30: note = Hydroxyproline
GPPGPPGPPGPPGVMGFPGPPGPPGPPGPPDLQYWYPIWDTH